(19) United States
(12) Patent Application Publication
(10) Pub. No.: US 2007/0213593 A1
(43) Pub. Date: Sep. 13, 2007

(54) Endoscope system

(75) Inventor: Masaya Nakaoka, Tokyo (JP)

Correspondence Address: SCULLY SCOTT MURPHY & PRESSER, PC, 400 GARDEN CITY PLAZA, SUITE 300, GARDEN CITY, NY, 11530, US

(73) Assignee: Olympus Corporation, Tokyo (JP)

(21) Appl. No.: 11/710,739

(22) Filed: Feb. 26, 2007

(30) Foreign Application Priority Data
Feb. 28, 2006 (JP) 2006-051914

(51) Int. Cl. A61B 1/06; A61B 6/00

(52) U.S. Cl. 600/181; 600/178; 600/160; 600/476

(57) ABSTRACT

The invention provides an endoscope which has a reduced-diameter insertion portion, which is capable of observation using a plurality of types of light with different spectral characteristics, and which can obtain high-resolution images. The invention provides an endoscope system comprising a light-source portion for emitting a plurality of types of irradiation light having different spectral characteristics, which are radiated towards an acquisition object; an optical system for transmitting the irradiation light towards the acquisition object; an image-acquisition portion, disposed in a portion inserted inside the body cavity and capable of acquiring fluorescence emitted from the acquisition object due to irradiation with the plurality of types of irradiation light, and light of a different wavelength band from the fluorescence; a variable-spectrum unit, disposed in a light path between the image-acquisition portion and a tip of the portion inserted inside the body cavity and capable of changing a wavelength band of light incident on the image-acquisition portion from the acquisition object by varying spectral characteristics thereof; and a control portion for controlling the light-source portion, the variable-spectrum unit, and the image-acquisition portion in association with the spectral characteristics of the irradiation light that the light-source portion emits, the spectral characteristics of the variable-spectrum unit, and a light exposure level of the image-acquisition portion.

### BACKGROUND OF THE INVENTION

1. Field of the Invention

The present invention relates to endoscope systems.

This application is based on Japanese Patent Application No. 2006-051914, the content of which is incorporated herein by reference.

2. Description of Related Art

In endoscopy of living organisms using endoscope systems, in order to observe the condition of the living organism with good resolution, it is preferable to carry out endoscopy using a plurality of types of light having different spectral characteristics.

Examples of endoscopes capable of carrying out observation using a plurality of types of light having different spectral characteristics are disclosed in the Publication of Japanese Patent No. 3683271 and Japanese Unexamined Patent Application, Publication No. 2001-190489.

In the endoscope disclosed in the Publication of Japanese Patent No. 3683271, in order to acquire a plurality of types of images using the plurality of types of light having different spectral characteristics, the light from an acquisition object is split into spectral components with dichroic mirrors. However, since it is difficult to integrate the dichroic mirrors in the tip of an insertion portion of the endoscope, the dichroic mirrors are provided as external units. Therefore, it is necessary to transmit light received at the tip of the insertion portion from the acquisition object to the external dichroic mirrors via an optical fiber bundle.

The endoscope disclosed in Japanese Unexamined Patent Application, Publication No. 2001-190489 does not use spectral means such as dichroic mirrors; instead, in order to allow observation of a plurality of types of light, a plurality of image-acquisition optical systems are disposed in the tip of an insertion portion of the endoscope.

However, in the case of the Publication of Japanese Patent No. 3683271, there is a drawback in that the resolution of the image acquired by the image-acquisition means depends on the number of optical fibers constituting the fiber bundle for transmitting this image. In other words, because the number of optical fibers that can be disposed inside the thin insertion portion of the endoscope is limited, when the diameter of the insertion portion is reduced, there is a problem in that it is difficult to perform high-resolution observation.

Furthermore, in the endoscope in Japanese Unexamined Patent Application, Publication No. 2001-190489, more space is required for disposing the plurality of image-acquisition optical systems; therefore, there is a problem in that it is difficult to reduce the diameter of the tip of the insertion portion of the endoscope.

In this endoscope, reflected light from the acquisition object is directly acquired by the image-acquisition means disposed in the tip of the insertion portion. However, in this endoscope, fluorescence is transmitted to external components using an optical fiber bundle, similarly to the Publication of Japanese Patent No. 3683271. Therefore, there is a problem in that it is not possible to perform high-resolution observation in this endoscope.

### BRIEF SUMMARY OF THE INVENTION

The present invention has been conceived in light of the circumstances described above, and an object thereof is to provide an endoscope which has a reduced-diameter insertion portion, which is capable of observation using a plurality of types of light with different spectral characteristics, and which can obtain high-resolution images.

In order to realize the objects described above, the present invention provides the following solutions.

According to a first aspect, the present invention provides an endoscope system, at least a portion of which is inserted into a body cavity of a living organism for acquiring images of an acquisition object inside the body cavity, comprising a light-source portion for emitting a plurality of types of irradiation light having different spectral characteristics, which are radiated towards the acquisition object; an optical system for transmitting the irradiation light from the light-source portion towards the acquisition object; an image-acquisition portion, disposed in the portion inserted inside the body cavity and capable of acquiring fluorescence emitted from the acquisition object due to irradiation with the plurality of types of irradiation light, and light of a different wavelength band from the fluorescence; a variable-spectrum unit, disposed in a light path between the image-acquisition portion and a tip of the portion inserted inside the body cavity and capable of changing a wavelength band of light incident on the image-acquisition portion from the acquisition object by varying spectral characteristics thereof; and a control portion for controlling the light-source portion, the variable-spectrum unit, and the image-acquisition portion in association with the spectral characteristics of the irradiation light that the light-source portion emits, the spectral characteristics of the variable-spectrum unit, and a light exposure level of the image-acquisition portion.

In the first aspect of the invention described above, fluorescence emitted from the acquisition object may be light emitted by exciting a fluorescent agent binding with a specific substance present in the acquisition object or a fluorescent agent accumulated in an organ of the living organism with one of the types of irradiation light serving as excitation light, and may be light in a band from red to near-infrared.

In the first aspect of the invention described above, the light of the different wavelength band from the fluorescence emitted from the acquisition object may be visible-band reflected light from the acquisition object.

In the first aspect of the invention described above, the light of the different wavelength band from the fluorescence emitted from the acquisition object may be visible-band light emitted by exciting a substance originally present in the acquisition object with one of the types of irradiation light serving as excitation light.

In the first aspect of the invention described above, the variable-spectrum unit may be configured so as to be capable of selectively switching between a first state in which the fluorescence emitted from the acquisition object is allowed to be incident on the image-acquisition portion and a second state in which the fluorescence emitted from the acquisition object is prevented from being incident on the image-acquisition portion.

In the first aspect of the invention described above, in the first and second states, the variable-spectrum unit may have a common transmission band in the spectral characteristic thereof.

In the first aspect of the invention described above, the common transmission band may include at least part of a green to blue band in a visible band formed of red, green, and blue.

In the first aspect of the invention described above, the control portion may put the variable-spectrum unit into the first state when the light-source portion emits irradiation light for generating the fluorescence from the acquisition object and may put the variable-spectrum unit into the second state when the light-source portion emits other irradiation light.

In the first aspect of the invention described above, the control portion may time-division switch among a plurality of types of irradiation light to be emitted from the light-source portion.

In the first aspect of the invention described above, the control portion may synchronously perform switching of the irradiation light emitted from the light source portion and switching of the spectral characteristic of the variable-spectrum unit.

In the first aspect of the invention described above, the control of the exposure level of the image-acquisition portion may be performed by light-level control (adjustment of the intensity or emission time of the irradiation light) of the light-source portion or exposure adjustment (adjustment of the shutter speed or aperture) of the image-acquisition portion in response to switching of the emitted irradiation light from the light-source portion.

The first aspect of the invention described above may further comprise an output portion for outputting image information for the image of the acquisition object acquired by the image-acquisition portion, wherein the control portion may subject the output image information from the output portion to processing in response to the switching of the emitted irradiation light from the light-source portion.

In the first aspect of the invention described above, the processing applied to the image information for the fluorescence image may be wavelength-conversion processing or color-conversion processing.

In the first aspect of the invention described above, the variable-spectrum unit may include optical members mutually opposing each other with a gap therebetween, and the spectral transmittance may be varied by changing the size of the gap between the optical members.

In the first aspect of the invention described above, a band of the reflected light may includes an optical absorption band of hemoglobin, and may be narrower than a band from green to blue, which is a part of a band comprised of red, green and blue bands in a spectrally sensitive band of the imaging-acquisition portion.

In the first aspect of the invention described above, the light-source portion is disposed outside the body cavity.

According to a second aspect, the present invention provides an endoscope system, at least a portion of which is inserted inside a body cavity of a living organism for acquiring images of an acquisition object inside the body cavity, comprising a light-source portion for emitting excitation light and illumination light having a different spectral characteristic from the excitation light, which are radiated towards the acquisition object; an optical system for transmitting the excitation light or the illumination light toward the acquisition object; an image-acquisition portion, disposed in the portion inserted inside the body cavity and capable of acquiring fluorescence emitted from the acquisition object, due to the excitation light, and reflected light of the illumination light reflected at the acquisition object; a variable-spectrum unit, disposed in a light path between the image-acquisition portion and a tip of the portion inserted inside the body cavity and capable of changing a wavelength of light incident on the image-acquisition portion from the acquisition object by varying the spectral characteristics thereof; and a control portion for controlling the light-source portion, the variable-spectrum unit, and the image-acquisition portion in association with the spectral characteristics of the excitation light and the illumination light that the light-source portion emits, the spectral characteristics of the variable-spectrum unit, and an exposure level of the image-acquisition portion.

In the second aspect of the present invention described above, the fluorescence may be light emitted by exciting, with the excitation light, a fluorescent agent which binds with a specific substance present in the acquisition object or which accumulates in an organ of the living organism, and may be light in a band from red to near-infrared.

In the second aspect of the present invention described above, the fluorescence may be light in the visible band emitted by exciting, with the excitation light, a substance originally present in the acquisition object.

In the second aspect of the present invention described above, the variable-spectrum unit may be configured to enable selective switching between a first state in which the fluorescence emitted from the acquisition object is allowed to be incident on the image-acquisition portion and a second state in which the fluorescence emitted from the acquisition object is prevented from being incident on the image-acquisition portion.

In the second aspect of the present invention described above, in the first and second states, the variable-spectrum unit may have a common transmission band in the spectral characteristics thereof.

In the second aspect of the present invention described above, the common transmission band may included at least part of a green to blue band in a visible band formed of red, green, and blue.

In the second aspect of the present invention described above, the control portion time-division may switch between the excitation light and the illumination light to be emitted from the light-source portion.

In the second aspect of the present invention described above, the variable-spectrum unit may include optical members mutually opposing each other with a gap therebetween, and the spectral transmittance may be varied by changing the size of the gap between the optical members.

In the second aspect of the present invention described above, the control of the exposure level of the image-acquisition portion by the control portion may be performed by light-level control (adjusting the intensity or the emission time of the irradiation light) of the light-source portion or exposure adjustment (adjusting the shutter speed or aperture) of the image-acquisition portion in response to the switching of the emitted irradiation light from the light-source portion.

The present invention affords advantages in that it is possible to reduce the diameter of the insertion portion of the endoscope while allowing a plurality of types of light with different spectral characteristics to be observed, and it is possible to acquire high-resolution images.

### BRIEF DESCRIPTION OF THE SEVERAL VIEWS OF THE DRAWINGS

FIG. 1 is a block diagram showing the overall configuration of an endoscope system according to a first embodiment of the present invention.

FIG. 2 is a diagram showing, in outline, the configuration of the interior of an image-acquisition unit of the endoscope system in FIG. 1.

FIG. 3 is a diagram showing transmittance characteristics of each optical element constituting the endoscope system in FIG. 1, as well as wavelength characteristics of irradiation light and fluorescence.

FIG. 4 is a timing chart for explaining the operation of the endoscope system in FIG. 1.

FIG. 5 is a timing chart showing an example of measurement mode switching during image acquisition.

FIG. 6 is a diagram showing the transmittance characteristic of each optical component constituting an endoscope system according to a second embodiment of the present invention, as well as wavelength characteristics of irradiation light and fluorescence.

FIG. 7 is a block diagram showing a light source unit in an endoscope system according to a third embodiment of the present invention.

FIG. 8 is a diagram showing the transmittance characteristic of each optical component constituting the endoscope system in FIG. 7, as well as wavelength characteristics of irradiation light and fluorescence.

FIG. 9 is a block diagram showing the overall configuration of an endoscope system according to a fourth embodiment of the present invention.

FIG. 10 is a front elevational view showing a rotating filter used in the endoscope system in FIG. 9.

FIG. 11 is a diagram showing the transmittance characteristic of each optical component constituting the endoscope system in FIG. 9.

FIG. 12 is a timing chart for explaining the operation of the endoscope system in FIG. 9.

### DETAILED DESCRIPTION OF THE INVENTION

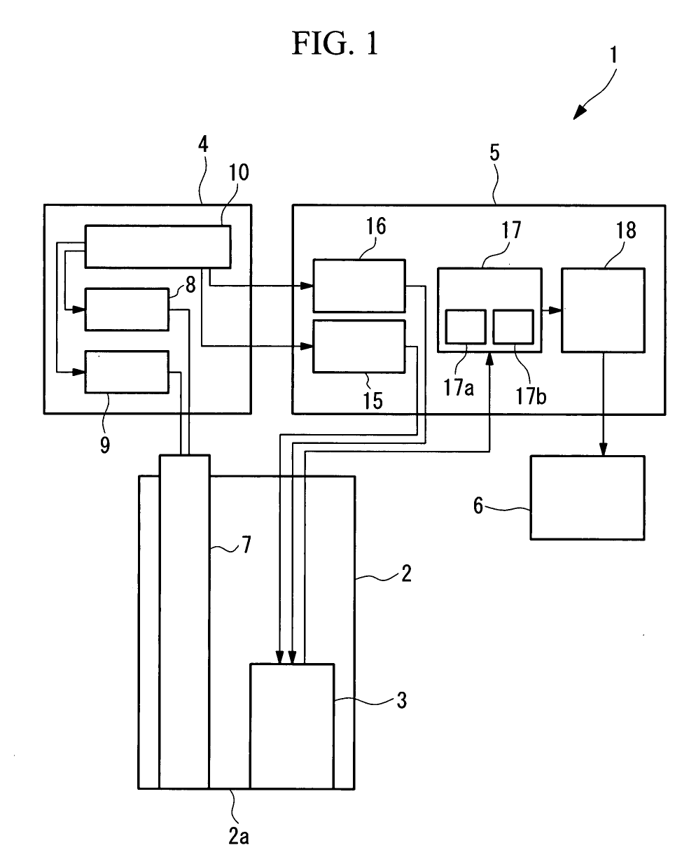

An endoscope system 1 according to a first embodiment of the present invention will be describe below with reference to FIGS. 1 to 4.

As shown in FIG. 1, the endoscope system 1 according to this embodiment includes an insertion portion 2 for insertion into a body cavity of a living organism, an image-acquisition unit (image-acquisition portion) 3 disposed inside the insertion portion 2, a light-source unit (light-source portion) 4 for emitting a plurality of types of light, a control unit (control portion) 5 for controlling the image-acquisition unit 3 and the light-source unit 4, and a display unit (output portion) 6 for displaying images acquired by the image-acquisition unit 3.

The insertion portion 2 has extremely narrow outer dimensions, allowing it to be inserted inside the body cavity of the living organism. The insertion portion 2 includes the image-acquisition unit 3 and a light guide (light-guiding optical system) 7 for transmitting light from the light-source unit 4 to a tip 2a.

The light-source unit 4 includes an illumination light source 8 which emits illumination light for illuminating an acquisition object inside the body cavity to obtain reflected light returning after reflection at the acquisition object, an excitation light source 9 which emits excitation light for irradiating the acquisition object inside the body cavity to generate fluorescence upon exciting a fluorescent material present inside the acquisition object, and a light-source control circuit 10 for controlling these light sources 8 and 9.

The illumination light source 8 is, for example, a combination of a xenon lamp and a bandpass filter (which are not shown in the drawing). The 50%-transmittance region of the bandpass filter is from 430 nm to 460 nm. In other words, the illumination light source 8 emits illumination light in the wavelength band of 430 nm to 460 nm.

The excitation light source 9 is, for example, a semiconductor laser emitting excitation light with a peak wavelength of 660±5 nm. Excitation light of this wavelength can excite fluorescent agents such as Cy5.5 (manufactured by Amersham) or ALEXAFLUOR700 (manufactured by Molecular Probes).

The light-source control circuit 10 is configured to alternately turn on and off the illumination light source 8 and the excitation light source 9 at a predetermined timing according to the timing chart described later.

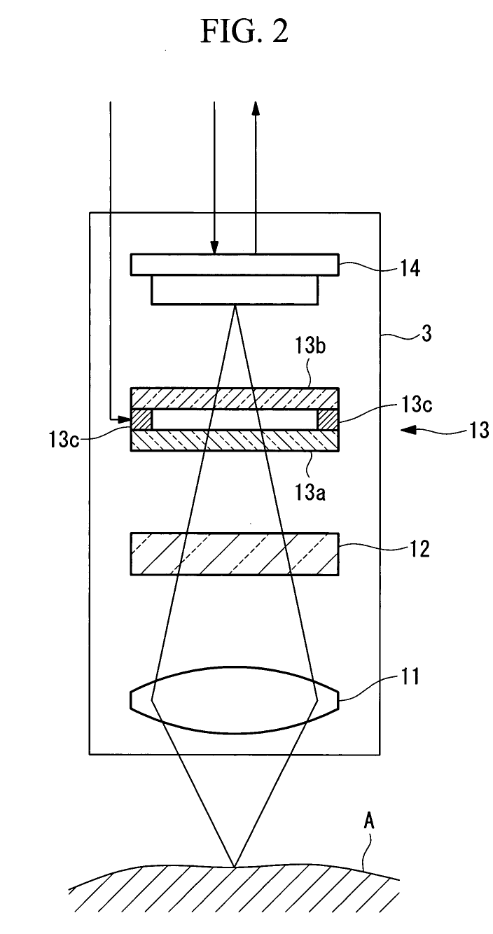

As shown in FIG. 2, the image-acquisition unit 3 includes an image-acquisition optical system 11 for focusing light incident from the acquisition object A, a excitation-light-cutting filter 12 for blocking the excitation light incident from the acquisition object A, a variable-spectrum device (variable-spectrum unit) 13 whose spectral characteristics can be changed by the operation of the control unit 5, and an image-acquisition device 14 for acquiring the light focused by the image-acquisition optical system 11 and converting it to an electrical signal.

The variable-spectrum device 13 is an etalon-type optical filter including two planar optical members 13a and 13b, which are disposed in parallel with a gap therebetween and in which reflective films are disposed on opposing faces thereof, and an actuator 13c for changing the gap between the optical members 13a and 13b. The actuator 13c is a piezoelectric device, for example. This variable-spectrum device 13 can change the wavelength band of the light transmitted therethrough by changing the size of the gap between the optical members 13a and 13b by operating the actuator 13c.

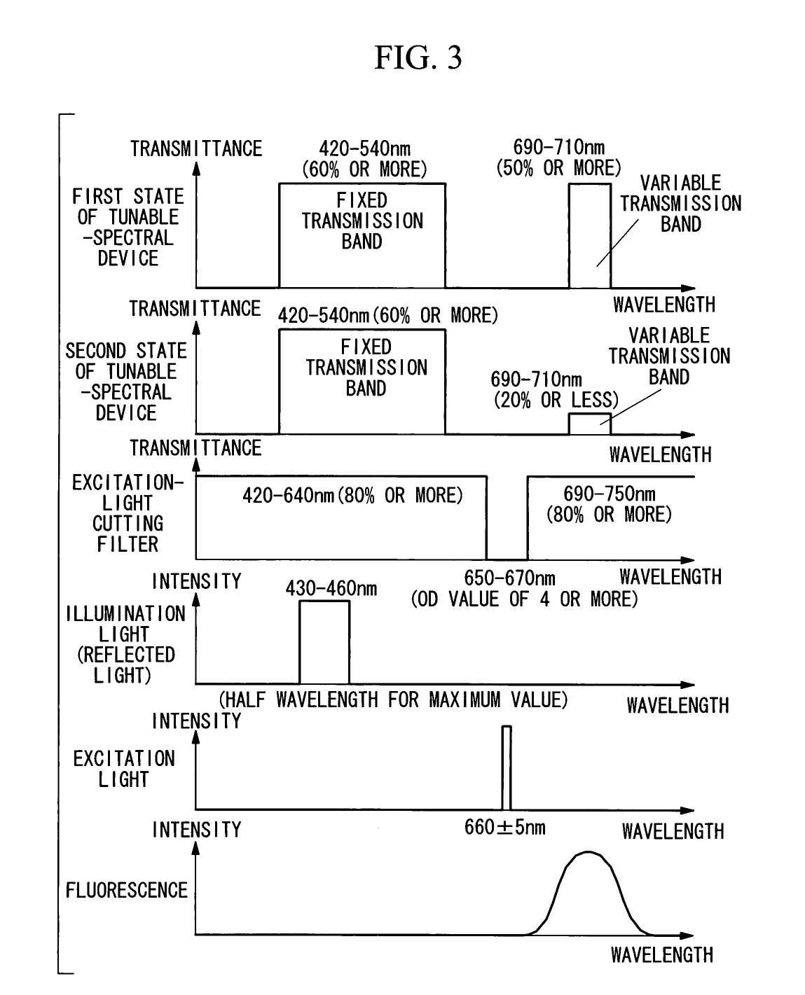

More specifically, as shown in FIG. 3, the variable-spectrum device 13 has a transmittance-versus-wavelength characteristic exhibiting two transmission bands, that is, one fixed transmission band and one variable transmission band. The fixed transmission band always transmits incident light, regardless of the state of the variable-spectrum device 13. The transmittance characteristic in the variable transmission band changes according to the state of the variable-spectrum device 13.

The variable-spectrum device 13 in this embodiment has a variable transmission band in a wavelength band (for example, 690 nm to 710 nm) including the wavelength of fluorescence (agent fluorescence) emitted by exciting a fluorescent agent with the excitation light. The variable-spectrum device 13 changes between two states according to a control signal from the control unit 5.

In the first state, the transmittance in the variable transmission band is increased to 50% or more to transmit the agent fluorescence. In the second state, the transmittance in the variable transmission band is reduced to 20% or less to block the agent fluorescence.

The agent fluorescence may also be blocked in the second state by changing the wavelengths of the variable transmission band from the first state.

The fixed transmission band of the variable-spectrum device 13 is located, for example, in the region from 420 nm to 540 nm. The transmittance of the fixed transmission band of the variable-spectrum device 13 is fixed at 60% or higher.

The fixed transmission band of the variable-spectrum device 13 is located in a wavelength band including the wavelength of the reflected illumination light. Accordingly, the variable-spectrum device 13 can transmit the reflected light towards the image-acquisition device 14 in both the first state and the second state.

The transmittance characteristic of the excitation-light-cutting filter 12 exhibits a transmittance of 80% or more in the wavelength band from 420 nm to 640 nm, an OD value of 4 or more (equivalent to a transmittance of 1×10⁻⁴ or less) in the wavelength band from 650 nm to 670 nm, and a transmittance of 80% or more in the wavelength band from 690 nm to 750 nm.

As shown in FIG. 1, the control unit 5 includes an image-acquisition-device driving circuit 15 for driving and controlling the image-acquisition device 14, a variable-spectrum-device control circuit 16 for driving and controlling the variable-spectrum device 13, a frame memory 17 for storing image information acquired by the image-acquisition device 14, and an image-processing circuit 18 for processing the image information stored in the frame memory 17 and outputting it to the display unit 6.

The image-acquisition-device driving circuit 15 and the variable-spectrum-device control circuit 16 are connected to the light-source control circuit 10. Accordingly, the image-acquisition-device driving circuit 15 and the variable-spectrum-device control circuit 16 drive and control the variable-spectrum device 13 and the image-acquisition device 14 in synchronization with the switching between the illumination light source 8 and the excitation light source 9 performed by the light-source control circuit 10.

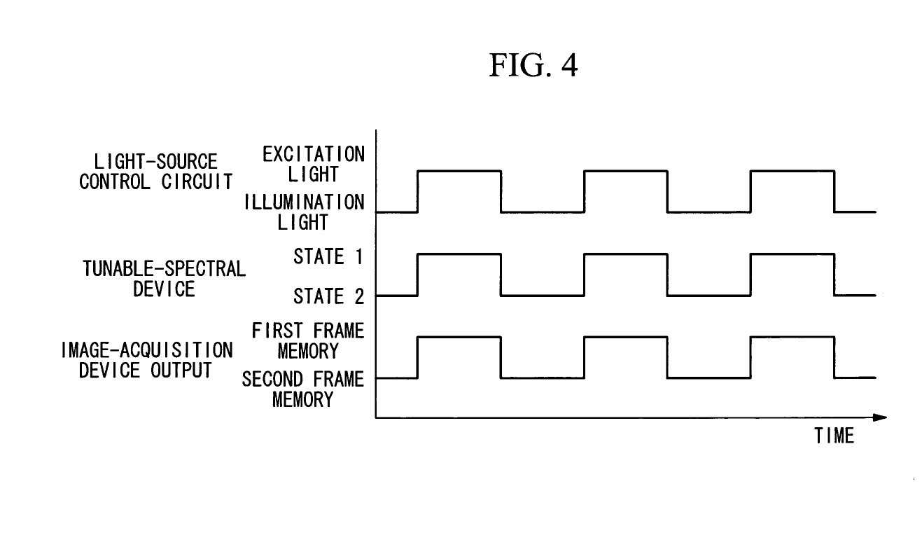

More concretely, as shown in the timing chart in FIG. 4, when the excitation light is emitted from the excitation light source 9 by operating the light-source control circuit 10, with the variable-spectrum-device control circuit 16 having set the variable-spectrum device 13 to the first state, the image-acquisition-device driving circuit 15 outputs to a first frame memory 17a the image information output from the image-acquisition device 14. Also, when the illumination light is emitted from the illumination light source 8, with the variable-spectrum-device control circuit 16 having set the variable-spectrum device 13 in the second state, the image-acquisition-device driving circuit 15 outputs to a second frame memory 17b the image information output from the image-acquisition device 14.

The image-processing circuit 18, for example, receives from the first frame memory 17a fluorescence image information acquired by irradiating excitation light and outputs it on the red channel of the display unit 6. Similarly, the image processing circuit 18 receives from the second frame memory 17b reflected-light image information acquired by radiating illumination light and outputs it on the green channel of the display unit 6.

The operation of the endoscope system 1 according to this embodiment, having such a configuration, will be described below.

To acquire an image of the acquisition object A inside the body cavity of the living organism using the endoscope system 1 according to this embodiment, with a fluorescent agent injected into the body, the insertion portion 2 is inserted into the body cavity so that the tip 2a thereof opposes the acquisition object A in the body cavity. In this state, the light-source unit 4 and the control unit 5 are operated, and by operating the light-source control circuit 10, the illumination light source 8 and the excitation light source 9 are alternately operated to generate illumination light and excitation light, respectively.

The excitation light and the illumination light generated in the light-source unit 4 are transmitted to the tip 2a of the insertion portion 2 via the light guide 7 and are radiated from the tip 2a of the insertion portion 2 towards the acquisition object A.

When the excitation light irradiates the acquisition object A, the fluorescent agent permeating the acquisition object A is excited and emits fluorescence. The fluorescence emitted from the acquisition object A is collected by the image-acquisition optical system 11 of the image-acquisition unit 3, passes through the excitation-light-cutting filter 12, and enters the variable-spectrum device 13.

By operating the variable-spectrum-device control circuit 16, the variable-spectrum device 13 is switched to the first state in synchronization with the operation of the excitation light source 9. Therefore, the transmittance of the variable-spectrum device 13 with respect to the fluorescence is increased, which allows the incident fluorescence to be transmitted therethrough. In this case, some of the excitation light irradiating the acquisition object A is reflected at the acquisition object A and enters the image-acquisition unit 3 together with the fluorescence. However, because the excitation-light-cutting filter 12 is provided in the image-acquisition unit 3, the excitation light is blocked and can thus be prevented from entering the image-acquisition device 14.

Then, the fluorescence passing through the variable-spectrum device 13 enters the image-acquisition device 14, where fluorescence image information is acquired. The acquired fluorescence image information is stored in the first frame memory 17a, is output to the red channel of the display unit 6 by the image processing circuit 18, and is displayed by the display unit 6.

On the other hand, when the illumination light irradiates the acquisition object A, the illumination light is reflected at the surface of the acquisition object A. The illumination light reflected by the acquisition object A is collected by the image-acquisition optical system 11, passes through the excitation-light-cutting filter 12, and enters the variable-spectrum device 13. Since the wavelength band of the reflected illumination light is located within the fixed transmission band of the variable-spectrum device 13, all of the reflected light entering the variable-spectrum device 13 is transmitted therethrough.

Then, the reflected light passing through the variable-spectrum device 13 enters the image-acquisition device 14, where reflected-light image information is acquired. The acquired reflected-light image information is stored in the second frame memory 17b, is output to the green channel of the display unit 6 by the image processing circuit 18, and is displayed by the display unit 6.

In this case, by operating the variable-spectrum-device control circuit 16, the variable-spectrum device 13 is switched to the second state in synchronization with the operation of the illumination light source 8. In other words, because the transmittance of the variable-spectrum device 13 with respect to the fluorescence is low in this case, even though the fluorescence is incident, it is blocked. Accordingly, only the reflected light is acquired by the image-acquisition device 14.

Hence, with the endoscope system 1 according to this embodiment, it is possible to provide the user with an image formed by combining the acquired fluorescence image and reflected-light image.

With the endoscope system 1 according to this embodiment, because the variable-spectrum device 13 whose optical transmittance characteristics are changed merely by changing the separation between the planar optical members 13a and 13b is used, it is possible to dispose the extremely compact variable-spectrum device 13 and the image-acquisition device 14 in the tip 2a of the insertion portion 2. Therefore, with the endoscope system 1 according to this embodiment, it is not necessary to extract the fluorescence or reflected light from the acquisition object A outside the body using a fiber bundle.

In the endoscope system 1 according to this embodiment, the state of the variable-spectrum device 13 is switched in synchronization with the switching of the multiple light sources 8 and 9 in the light-source unit 4; therefore, it is possible to acquire light of a plurality of different wavelength bands using the same image-acquisition device 14. Accordingly, in the endoscope system 1 according to this embodiment, it is not necessary to provide a plurality of image-acquisition optical systems corresponding to the fluorescence and the reflected light. As a result, it is possible to reduce the diameter of the insertion portion 2 in the endoscope system 1 according to this embodiment.

Because of the presence of external light that is transmitted through the organs, even though they are inside the body cavity of the living organism, it is important to reduce noise when observing extremely weak light, particularly in fluorescence observation. In the endoscope system 1 according to this embodiment, by providing the variable-spectrum device 13 in the image-acquisition unit 3, it is possible to always block light of wavelengths other than the acquisition object, even if the wavelength band being observed changes. Therefore, it is possible to acquire superior images with low noise.

In the endoscope system 1 according to this embodiment, the illumination light source 8 generates illumination light in the wavelength band of 430 nm to 460 nm. Because this wavelength band includes the absorption band of hemoglobin, it is possible to acquire information about the structure of blood vessels and so on that are comparatively close to the surface of the living organism by acquiring the reflected light.

Commercially available fluorescent agents such as Cy5.5 and ALEXAFLUOR700 emit fluorescence in the near-infrared band upon absorbing red excitation light. From these fluorescent agents, it is possible to produce fluorescent probes which emit light when binding with substances inside living organisms. When a fluorescent probe which binds with a substance related to disease or whose level of accumulation in an internal organ changes due to disease is produced and administered to a living organism, it is possible to obtain information about the disease by acquiring this fluorescence.

Generally, the effect of scattering in a living organism decreases as the wavelength increases, and it is easy to observe even fluorescence generated deep inside a living organism. However, light with a wavelength of 1 μm or more is decreased due to absorption by water, which makes its observation difficult. Therefore, by using a fluorescent agent which emits fluorescence in the near-infrared band, as in the endoscope system 1 according to this embodiment, it is possible to effectively obtain information about the inside of the living organism, particularly information about disease, such as cancer, originating from the vicinity of mucus membranes.

In the endoscope system 1 according to this embodiment, in the image-acquisition unit 3, the image-acquisition optical system 11, the excitation-light-cutting filter 12, and the variable-spectrum device 13 are arranged in this order from the tip 2a side of the insertion portion 2. However, the order in which these components are arranged is not limited to this order; it is possible to arrange them in any order.

In general, when acquiring an image inside the body cavity of a living organism, the resolution of the agent-fluorescence image is extremely low compared to the resolution of the reflected-light image. As a result, it is considered necessary to appropriately adjust the level of light (exposure level) incident on the image-acquisition device 14 as one switches between observing the reflected-light image and observing the agent-fluorescence image.

Therefore, in order to operate the fluorescence endoscope system described above according to the brightness of the image measured with the image-acquisition device 14 to adjust the image brightness to approach a predetermined target value set in advance, it is preferable that the control unit 5 switch the irradiation light (excitation light) from the light-source unit 4 and the spectral characteristics of the variable-spectrum device 13 and, in addition, perform adjustment of the exposure level of the image-acquisition unit 3 (the image-acquisition device 14) during image acquisition. More specifically, in order to adjust this exposure level, it is preferable to perform one or a plurality of adjustments from among light-level control (adjusting the light emission intensity or light emission time) of the illumination light (excitation light) from the light-source unit 4 and adjustment of the exposure (adjusting the shutter speed or aperture) of the image-acquisition unit 5 or the gain of the image-acquisition unit 5.

Such adjustment is particularly important when constructing a single image from a plurality of images with very different brightnesses and high-brightness regions (bright regions), such as when combining a reflected-light image, which is comparatively bright over the entire image, and a agent-fluorescence image, in which the fluorescence region is limited to the region where the agent is applied (administered).

The image brightness measured during this image-brightness adjustment may be a value measured in a mode where the average value of the entire image or a portion thereof defines the image brightness, that is, an average light-measuring mode, or it may be a value measured in a mode where the maximum value of the entire image or a partial region thereof defines the image brightness, that is, a peak light-measuring mode.

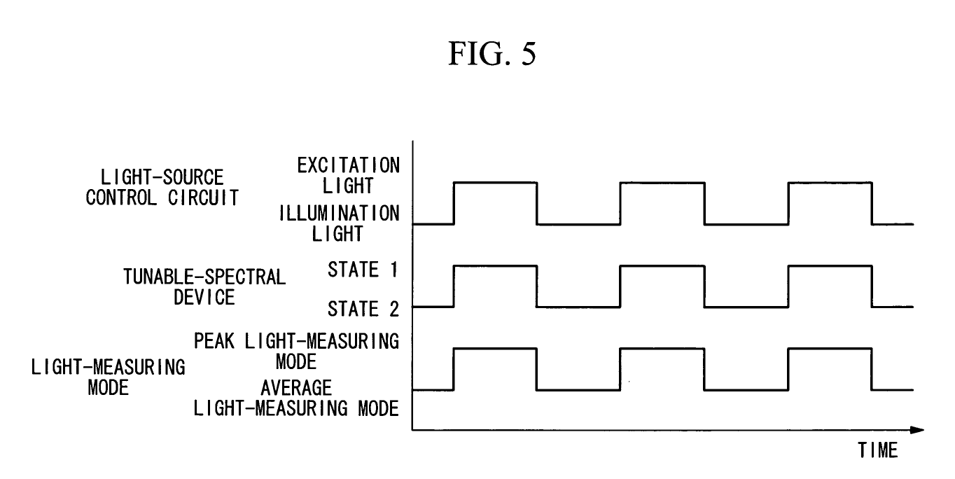

The mode for measuring the image brightness may be controlled in conjunction with the light-source control circuit and the variable-spectrum-device control circuit so as to enter the average light-measuring mode during reflected-light image acquisition and the peak light-measuring mode during agent-fluorescence image acquisition, with a predetermined timing according to the timing chart shown in FIG. 5.

The reason for this is that, during reflected-light image acquisition, there are many instances where the subject to be acquired appears in the entire image, forming a comparatively bright region over the entire image, and therefore, the average light-measuring mode is more effective. If peak light measurement were performed on such a reflected-light image, brightness adjustment should be carried out to make the very bright region, for example, reflection from mucus in the living organism, approach a target value, causing the acquisition object to become dark.

On the other hand, during agent-fluorescence image acquisition, in many instances the generation of fluorescence is limited only to the region where the fluorescent agent is administered (applied), causing most of the image to be a dark region where no fluorescence is generated, forming an image in which the agent fluorescence is visible only in a portion of the image; therefore, the peak light-measurement mode is more effective.

If average light measurement were carried out for such a fluorescence image, brightness adjustment should be carried out to make the dark region occupying most of the image approach the target brightness. Therefore, noise in regions where no fluorescence is generated would be emphasized, producing an image that is difficult to observe.

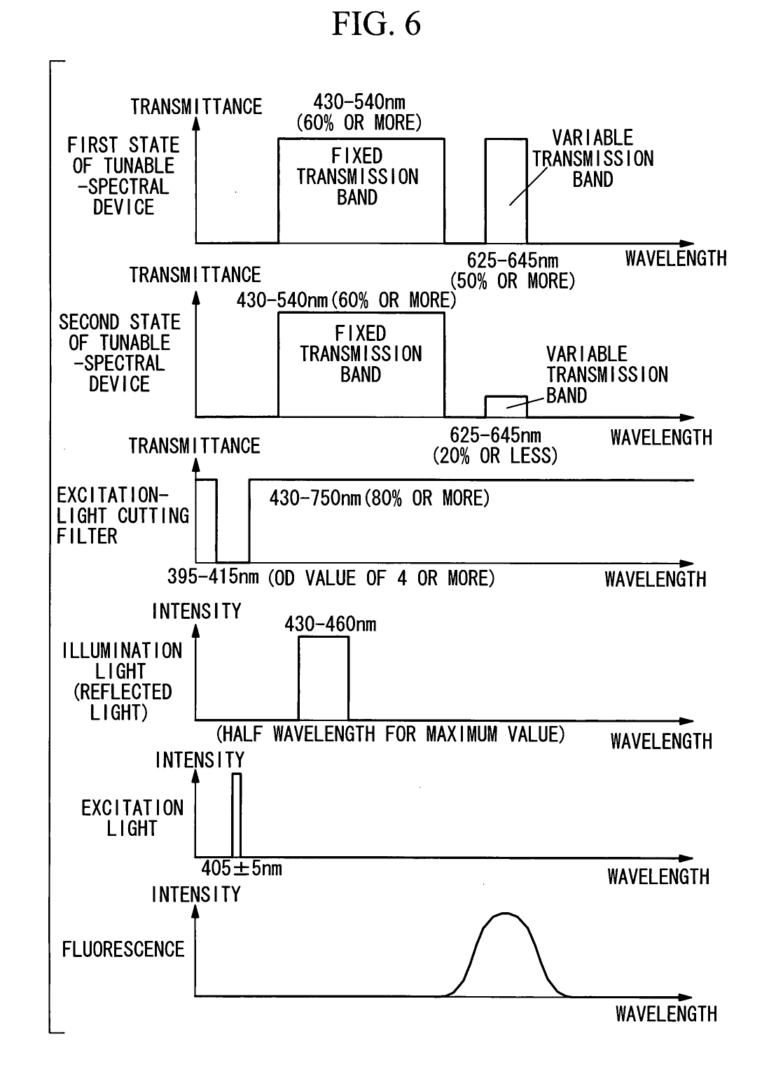

Next, an endoscope system according to a second embodiment of the present invention will be described below with reference to FIG. 6.

In the description of this embodiment, parts having the same configuration as those in the endoscope system 1 according to the first embodiment described above are assigned the same reference numerals, and a description thereof shall be omitted here.

As shown in FIG. 6, in the endoscope system according to this embodiment, the wavelength of the excitation light emitted from the excitation light source 9 differs from that in the endoscope system 1 according to the first embodiment. Based on this, in the endoscope system according to this embodiment, the transmittance characteristics of the variable-spectrum device 13 and the excitation-light-cutting filter 12 differ from those in the endoscope system 1 according to the first embodiment.

In the endoscope system according to this embodiment, a semiconductor laser having a peak wavelength of 405±5 nm is used as the excitation light source 9. The excitation light of this wavelength can excite autofluorescent material such as porphyrin originally present in the living organism.

Similarly to the first embodiment, the variable-spectrum device 13 has a fixed transmission band including the wavelength band of reflected light and a variable transmission band for switching between a first state in which the transmittance is high at the wavelengths of the autofluorescence and a second state in which the transmittance is low at the wavelengths of the autofluorescence.

The fixed transmission band of the variable-spectrum device 13 is, for example, a wavelength band of 430 nm to 540 nm. The variable-spectrum device 13 has a transmittance of 60% or greater in the fixed transmission band. The variable transmission band of the variable-spectrum device 13 is a wavelength band from 625 nm to 645 nm. The variable transmission band of the variable-spectrum device 13 has a transmittance of 50% or more in the first state and a transmittance of 20% or less in the second state.

The transmittance characteristic of the excitation-light-cutting filter 12 has an OD value of 4 or more (a transmittance of 1×10⁻⁴ or less) in the wavelength band from 395 nm to 415 nm and a transmittance of 80% or more in the wavelength band from 430 nm to 750 nm.

With the endoscope system according to this embodiment, having such a configuration, when excitation light is emitted from the excitation light source 9 by operating the light-source-control circuit 10, the operation of the illumination light source 8 is stopped, and the acquisition object A is irradiated with only the excitation light. At this time, the variable-spectrum device 13 is switched to the first state by the variable-spectrum-device control circuit 16, in synchronization with the operation of the excitation light source 9. Therefore, the autofluorescence generated in the acquisition object A is transmitted through the variable-spectrum device 13, is acquired by the image-acquisition device 14, and is stored in the first frame memory 17a.

On the other hand, when the illumination light is emitted from the illumination light source 8 by operating the light-source control circuit 10, the operation of the excitation light source 9 is stopped, and the acquisition object A is irradiated with only the illumination light. At this time, the variable-spectrum device 13 is switched to the second state by the variable-spectrum-device control circuit 16, in synchronization with the operation of the illumination light source 8. Therefore, the reflected light from the acquisition object A is transmitted through the variable-spectrum device 13, is acquired by the image-acquisition device 14, and is stored in the second frame memory 17b.

The maximum excitation wavelength of many autofluorescent materials is short, such as the ultraviolet region, and excitation in regions such as green and red is therefore difficult because they are outside the excitation wavelength region of the autofluorescent materials. In contrast, ultraviolet light is easily scattered in living organisms, and except for regions extremely close to the surface of the living organism, it is difficult for the excitation light to reach the autofluorescent materials. Therefore, with the endoscope system according to this embodiment, by using a semiconductor laser with a peak wavelength of 405±5 nm in the excitation light source 9, for diagnosis, it is possible to excite autofluorescent material existing at the required depth using blue excitation light.

The fluorescence from porphyrin, which is one of the autofluorescent materials inside living organisms, has a peak wavelength close to 630 nm, and it is known that the intensity thereof changes due to disease. Therefore, by observing a fluorescent image of the autofluorescence in a band including the wavelength of 630 nm, it is possible to obtain information related to the disease.

Similarly to the endoscope system according to the first embodiment, the illumination light source 8 emits illumination light in a wavelength band from 430 nm to 460 nm. Because this wavelength band includes the absorption band of hemoglobin, acquiring an image of the reflected light of this illumination light allows to obtain information about the structure and so forth of blood vessels comparatively close to the surface of the living organism to be obtained.

In general, when acquiring an image of the interior of a body cavity of a living organism, the brightness of the autofluorescence image of the living organism is extremely small compared to the brightness of the reflected-light image. As a result, it is considered necessary to appropriately adjust the amount of light (exposure level) incident on the image-acquisition device 14 every time a reflected-light image or an autofluorescence image is acquired.

Therefore, in order to operate the fluorescence endoscope system described above according to the image brightness measured by the image-acquisition device 14 to perform image-brightness adjustment to make the image brightness approach a predetermined target value which is determined in advance, it is preferable that the control unit 5 switch the irradiation light (excitation light) from the light-source unit 4 and the spectral characteristics of the variable-spectrum device 13 and, in addition, that it perform exposure adjustment of the image-acquisition unit 3 (image-acquisition device 14) during image acquisition. More concretely, in order to adjust the exposure level, it is preferable to perform one or a plurality of adjustments from among light-level control (adjustment of the emission intensity or the emission time) of the illumination (excitation) light from the light source 4 and adjustment of the exposure (adjustment of the shutter speed or aperture) of the image-acquisition unit 5 or the gain of the image-acquisition unit 5.

Such adjustment is particularly important when constructing a single image from a plurality of images with very different brightnesses, such as when combining the reflected-light image, which is comparatively bright over the entire image, and the autofluorescence image, which is weak.

The image brightness measured during this image-brightness adjustment may be a value measured in a mode where the average value of the entire image or a portion thereof defines the image brightness, that is, an average light-measuring mode, or it may be a value measured in a mode where the maximum value in the entire image or in a partial region thereof defines the image brightness, that is, a peak light-measuring mode.

During reflected-light image acquisition, the mode for setting the image brightness, such as the average light measurement mode, may also be controlled in association with the light-source control circuit and the variable-spectrum-device control circuit with a predetermined timing.

The reason for this is that, during reflected-light image acquisition, there are many instances where the subject to be acquired appears in the entire image, and the average light-measuring mode is thus more effective. If peak light measurement were performed on such a reflected-light image, brightness adjustment should be carried out to make the very bright region, for example, reflection from mucus in the living organism, approach a target value, causing the acquisition object to become dark.

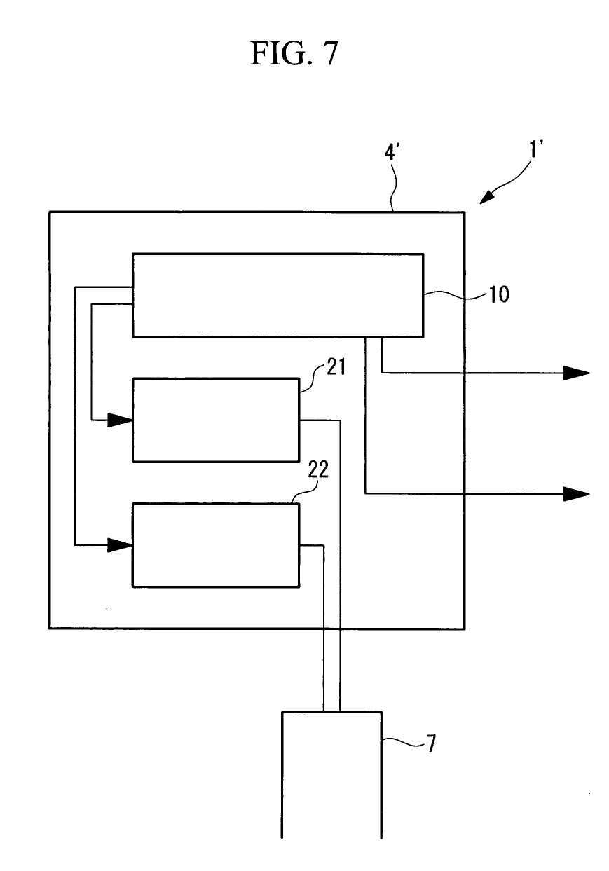

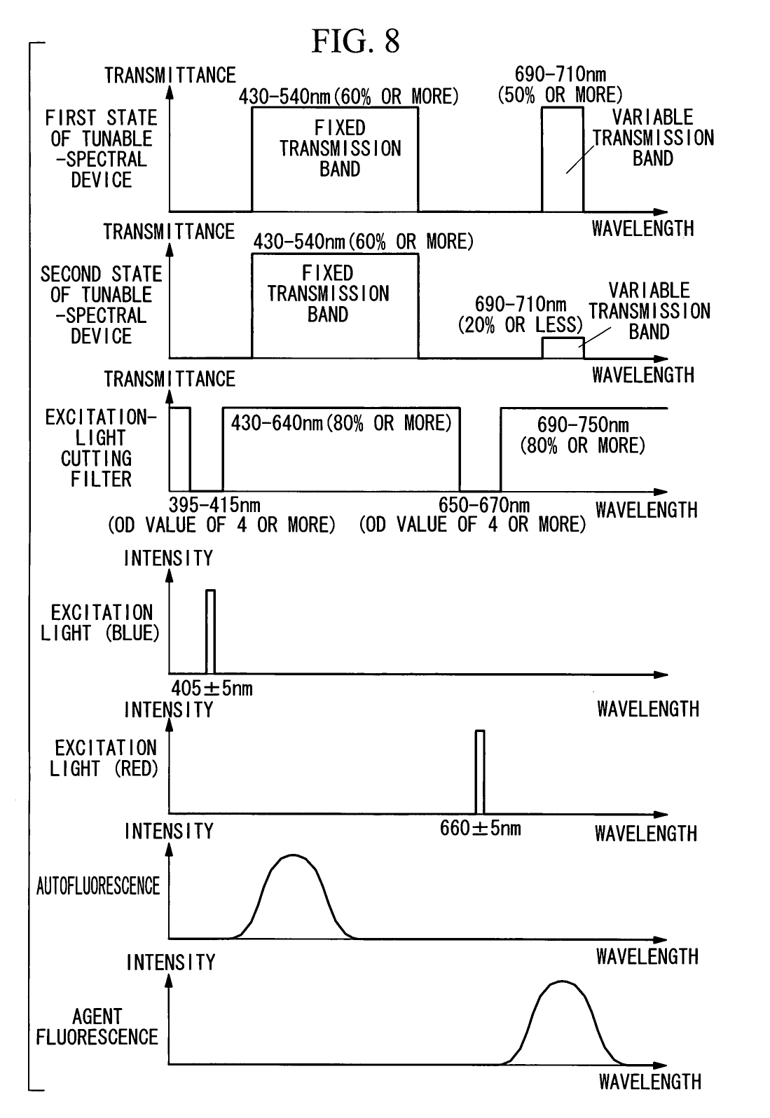

Next, an endoscope system 1′ according to a third embodiment of the present invention will be described with reference to FIGS. 7 and 8.

In the description of this embodiment, parts having the same configuration as those in the endoscope system 1 according to the first embodiment described above are assigned the same reference numerals, and a description thereof is omitted here.

The endoscope system 1′ according to this embodiment differs from the endoscope system 1 according to the first embodiment in the configuration of a light-source unit 4′ and the transmittance characteristics of the variable-spectrum device 13 and the excitation-light-cutting filter 12.

As shown in FIG. 7, the light-source unit 4′ of the endoscope system 1′ according to this embodiment includes two excitation light sources 21 and 22.

The first excitation light source 21 is a semiconductor laser emitting first excitation light with a peak wavelength of 660±5 nm. It is possible to excite a fluorescent agent such as Cy5.5 or ALEXAFLUOR700 with the first excitation light which this semiconductor laser emits.

The second excitation light source 22 is a semiconductor laser emitting second excitation light with a peak wavelength of 405±5 nm. It is possible to excite autofluorescence of collagen, NADH, FAD, and the like in the living organism with the second excitation light of this wavelength.

As shown in FIG. 8, the variable-spectrum device 13 has a fixed transmission band including the wavelength band of the autofluorescence and a variable transmission band for switching between a first state in which the transmittance at the wavelengths of the agent fluorescence is high and a second state in which the transmittance at the wavelengths of the agent fluorescence is low.

The fixed transmission band has a transmittance of 60% or more in, for example, a wavelength band of 430 nm to 540 nm. The variable transmission band covers a wavelength band of 690 nm to 710 nm. The variable transmission band of the variable-spectrum device 13 has a transmittance of 50% or more in the first state and a transmittance of 20% or less in the second state.

The transmittance characteristics of the excitation-light-cutting filter 12 exhibit an OD value of 4 or more (a transmittance of 1×10⁻⁴ or less) in the wavelength band of 395 nm to 415 nm, a transmittance of 80% or more in the wavelength band of 430 nm to 640 nm, an OD value of 4 or more (1×10⁻⁴ or less) in the wavelength band of 650 nm to 670 nm, and a transmittance of 80% or more in the wavelength band of 690 nm to 750 nm.

With the endoscope system 1′ according to this embodiment, having such a configuration, when excitation light is emitted from the first excitation light source 21 by operating the light-source control circuit 10, the operation of the second excitation light source 22 is stopped, and only the first excitation light irradiates the acquisition object A. At this time, the variable-spectrum device 13 is switched to the first state by the variable-spectrum-device control circuit 16, in synchronization with the operation of the first excitation light source 21. Therefore, agent fluorescence generated at the acquisition object A is transmitted through the variable-spectrum device 13 and is acquired by the image-acquisition device 14, and agent-fluorescence image information is stored in the first frame memory 17a.

On the other hand, when the second excitation light is emitted from the second excitation light source 22 by operating the light-source control circuit 10, the operation of the first excitation light source 21 is stopped, and only the second excitation light irradiates the acquisition object A. At this time, the variable-spectrum device 13 is switched to the second state by the variable-spectrum-device control circuit 16, in synchronization with the operation of the second excitation light source 22. Therefore, the autofluorescence generated at the acquisition object A is transmitted through the variable-spectrum device 13 and is acquired by the image-acquisition device 14, and autofluorescence image information is stored in the second frame memory 17b.

The agent-fluorescence image information stored in the first frame memory 17a is output by the image processing circuit 18 on, for example, the red channel of the display unit 6 and is displayed by the display unit 6.

On the other hand, the autofluorescence image information stored in the second frame memory 17b is output by the image-processing circuit 18 on, for example, the green channel of the display unit 6 and is displayed by the display unit 6. Accordingly, it is possible to provide the fluorescence endoscope system 1′ that presents the user with a combined image formed by combining the agent-fluorescence image and the autofluorescence image and that acquires a plurality of images carrying different types of information.

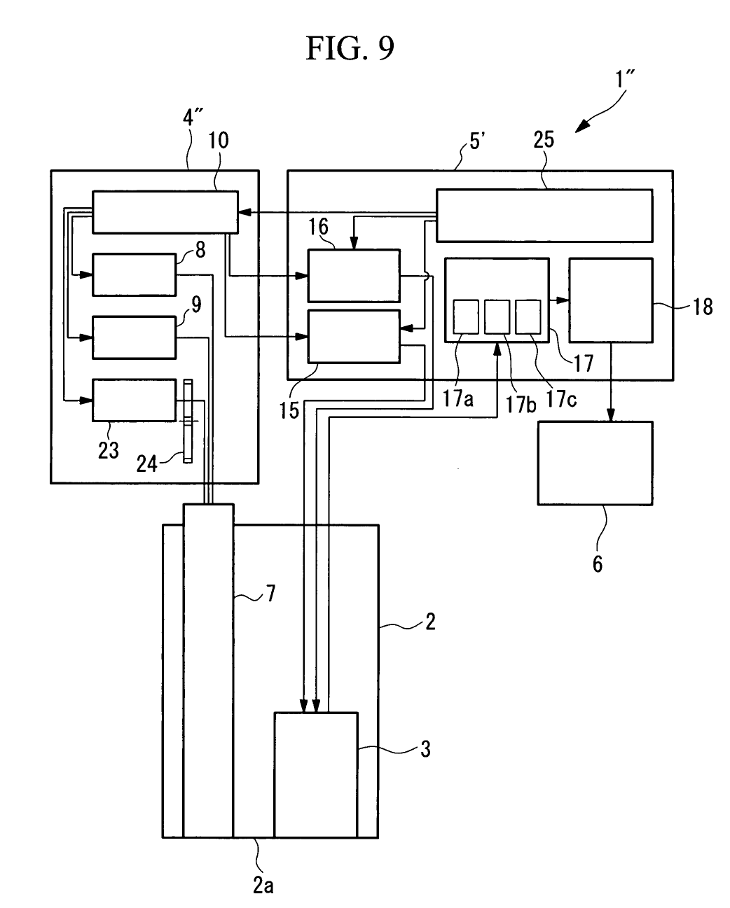

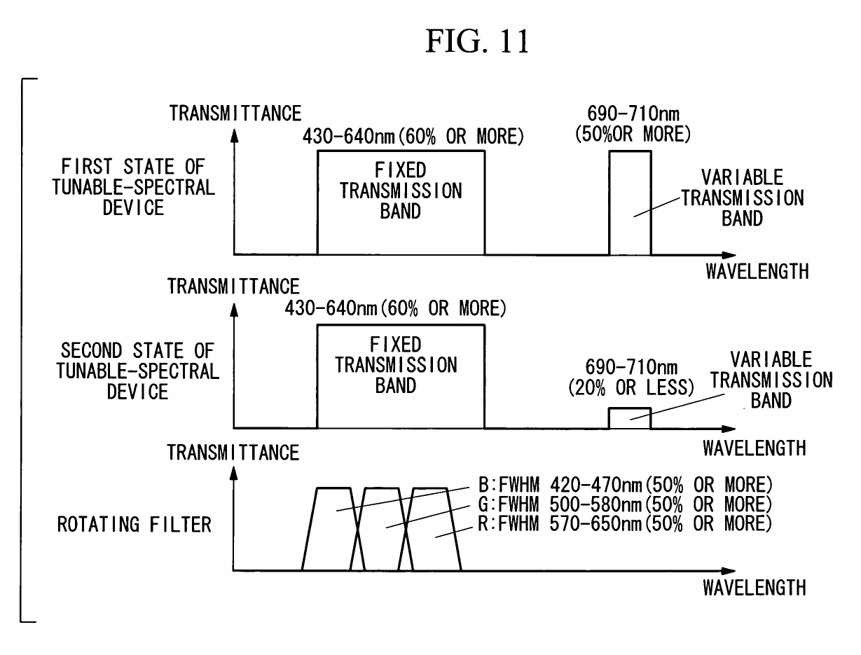

Next, an endoscope system 1″ according to a fourth embodiment of the present invention will be described with reference to FIGS. 9 to 12.

In the description of this embodiment, parts having the same configuration as those in the endoscope system 1 according to the first embodiment described above are assigned the same reference numerals, and a description thereof is omitted here.

The endoscope system 1″ according to this embodiment differs from the endoscope system 1 according to the first embodiment described above in a control unit 51 and in the configuration of a light source unit 4″.

As shown in FIG. 9, the light-source unit 4″ of the endoscope system 1″ according to this embodiment includes a normal light source 23, in addition to the illumination light source 8 and the excitation light source 9. These light sources 8, 9, and 23 are controlled so as to be turned on and off by the light-source control circuit 10.

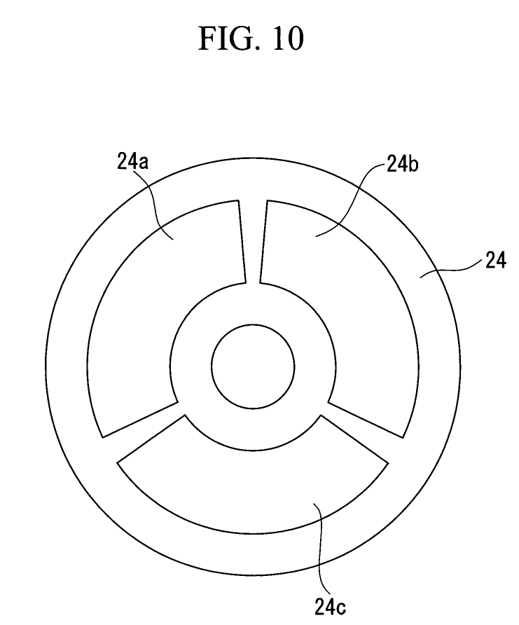

The normal light source 23 emits illumination light in a wavelength band of 420 nm to 650 nm. The normal light source 23 includes a rotating filter 24 in the light path to the light guide 7. As shown in FIG. 10, the rotating filter 24 includes individual R, G, and B filters 24a, 24b, and 24c. By rotating the rotating filter 24, it is possible to sequentially emit red light (R), green light (G), or blue light (B) towards the light guide 7.

The spectral transmittance characteristic of the R filter 24a has a transmittance of 50% or more in the wavelength band of 570 nm to 650 nm and a transmittance of 20% or less at wavelengths outside this range.

The spectral transmittance characteristic of the G filter 24b has a transmittance of 50% or more in a wavelength band of 500 nm to 580 nm and a transmittance of 20% or less at wavelengths outside this range.

The spectral transmittance characteristic of the B filter 24c has a transmittance of 50% or more in the wavelength band of 420 nm to 470 nm and a transmittance of 20% or less at wavelengths outside this range.

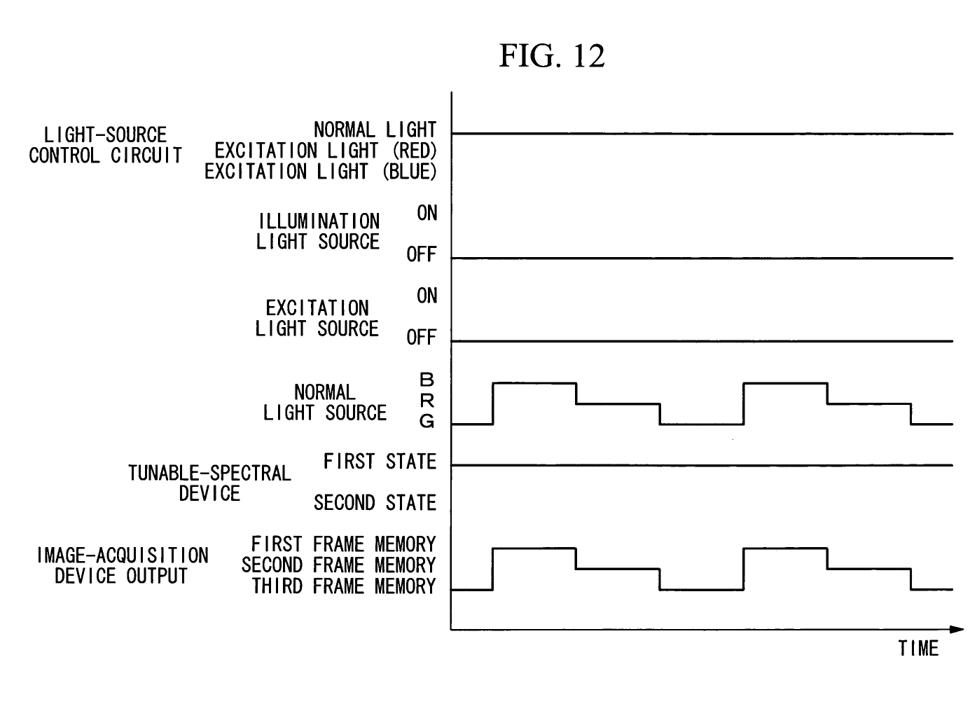

An observation-mode selection circuit 25 is provided in the control unit 5′. The user, by operating this observation-mode selection circuit 25, can selectively switch between a fluorescence observation mode and a normal-light observation mode. When the normal-light observation mode is selected in the observation-mode selection circuit 25, as shown in FIG. 12, by the operation of the light-source control circuit 10, the illumination light source 8 and the excitation light source 9 are turned off, and the normal light source 23 is turned on.

When the normal-light observation mode is selected, the variable-spectrum device 13 is set in either the first or the second state.

Moreover, when the normal-light observation mode is selected, by operating the image-acquisition-device driving circuit 15, the image information output from the image-acquisition device 14 corresponding to illumination with each color of light, that is, R, G, and B, is stored in first to third frame memories 17a, 17b, and 17c, respectively.

Then, in the normal-light observation mode, the image processing circuit 18 generates a normal-light image from the reflected light images stored in the first, second, and third frame memories 17a, 17b, and 17c and outputs them on the display unit 6.

The operation in the fluorescence observation mode is the same as in the first embodiment.

When observing the agent fluorescence, it is necessary to administer the fluorescent agent to the living organism before performing fluorescence observation. However, when administering the agent orally, intravenously, and so forth, it is necessary to administer a large amount of the fluorescent agent, which may result in the problem of consumption of a large amount of fluorescent agent, which is generally expensive. Therefore, as the method of administration, it is preferable to administer the agent locally by spraying it under endoscopy.

However, because the intensity of the fluorescence is usually very weak, the quality of the fluorescence image tends to deteriorate due to noise and so forth. Therefore, cases where it is not possible to sufficiently identify the affected area only by fluorescence observation, making it difficult to spray the fluorescent agent, should also be considered. In addition, in comparing the conventional endoscope image and the fluorescence image, there is also a problem in that it is difficult to check for changes in the affected area.

With the endoscope system 1″ according to this embodiment, because it has a normal observation mode for observing only reflected light in the visible wavelength band outside that used for fluorescence observation, it is possible to selectively switch between the normal observation mode and the fluorescence observation mode based on the user's operation as required. Therefore, by switching to the normal observation mode when spraying the fluorescent agent and to the fluorescence observation mode when performing fluorescence observation, an advantage is afforded in that it is possible to easily confirm when spraying the fluorescent agent and it is possible to easily obtain information about the disease. In addition, because the observation method is the same as with a conventional endoscope image, comparison with the conventional endoscope image is facilitated.

In order to prevent erroneous use or complicated operations by the user, it is preferable to automatically set the normal observation mode when the power is turned on.

The fluorescence endoscope systems 1, 1′, and 1″ of the present invention are not limited to scopes of the type having the image-acquisition device 14 at the tip of the insertion portion 2 to be inserted inside the body cavity of the living organism. They may also be applied to capsule-type endoscopes in which the light-source portion, the image-acquisition portion, and the variable-spectrum unit are provided in a single housing and the entire housing can be inserted inside the body cavity of the living organism.

1. An endoscope system, at least a portion of which is inserted into a body cavity of a living organism for acquiring images of an acquisition object inside the body cavity, comprising:
a light-source portion configured to emit a plurality of types of irradiation light having different spectral characteristics, which are radiated towards the acquisition object;
an optical system configured to transmit the irradiation light from the light-source portion towards the acquisition object;
an image-acquisition portion, disposed in the portion inserted inside the body cavity and capable of acquiring fluorescence emitted from the acquisition object due to irradiation with the plurality of types of irradiation light, and light of a different wavelength band from the fluorescence;
a variable-spectrum unit, disposed in a light path between the image acquisition portion and a tip of the portion inserted inside the body cavity and capable of changing a wavelength band of light incident on the image-acquisition portion from the acquisition object by varying spectral characteristics thereof; and
a control portion configured to control the light-source portion, the variable-spec unit, and the image-acquisition portion in association with the spectral characteristics of the irradiation light that the light-source portion emits, the spectral characterstics of the variable-spectrum unit, and a light exposure level of the image-acquisition portion.

2. An endoscope system according to claim 1, wherein the fluorescence emitted from the acquisition object is light emitted by exciting a fluorescent agent binding with a specific substance present in the acquisition object or a fluorescent agent accumulated in an organ of the living organism with one of the types of irradiation light serving as excitation light, and is light in a band from red to near-infrared.

3. An endoscope system according to claim 1, wherein the light of a different wavelength band from the fluorescence emitted from the acquisition object is visible-band reflected light from the acquisition object.

4. An endoscope system according to claim 1, wherein the light of a different wavelength band from the fluorescence emitted from the acquisition object is visible-band light emitted by exciting a substance originally present in the acquisition object with one of the types of irradiation light serving as excitation light.

5. An endoscope system according to claim 1, wherein the variable-spectrum unit is configured so as to be capable of selectively switching between a first state in which the fluorescence emitted from the acquisition object is allowed to be incident on the image-acquisition portion and a second state in which the fluorescence emitted from the acquisition object is prevented from being incident on the image-acquisition portion.

6. An endoscope system according to claim 5, wherein, in the first and second states, the variable-spectrum unit has a common transmission band in the spectral characteristic thereof.

7. An endoscope system according to claim 6, wherein the common transmission band includes at least part of a green to blue band in a visible band formed of red, green, and blue.

8. An endoscope system according to claim 5, wherein the control portion puts the variable-spectrum unit into the first state when the light-source portion emits irradiation light for generating the fluorescence from the acquisition object and puts the variable-spectrum unit into the second state when the light-source portion emits other irradiation light.

9. An endoscope system according to claim 1, wherein the control portion time-division switches among a plurality of types of irradiation light to be emitted from the light-source portion.

10. An endoscope system according to claim 1, wherein when the control portion synchronizes switching of the irradiation light emitted from the light source portion and switching of the spectral characteristic of the variable-spectrum unit.

11. An endoscope system according to claim 1, wherein the control of the exposure level of the image-acquisition portion is performed by light-level control of the light-source portion or exposure adjustment of the image-acquisition portion in response to switching of the emitted irradiation light from the light-source portion.

12. An endoscope system according to claim 1, further comprising:
an output portion configured to output image information for the image of the acquisition object acquired by the image-acquisition portion,
wherein the control portion subjects the output image information from the output portion to processing in response to the switching of the emitted irradiation light from the light-source portio

13. An endoscope system according to claim 12, wherein the processing applied to the image information for the fluorescence image is wavelength-conversion processing or color-conversion processing.

14. An endoscope system according to claim 1, wherein the variable-spectrum unit includes optical members mutually opposing each other with a gap therebetween, and the spectral transmittance is varied by changing the size of the gap between the optical members.

15. An endoscope system according to claim 3, wherein a band of the visible band reflected light includes an optical absorption band of hemoglobin, and is narrower than a band from green to blue, which is a part of a band comprised of red, green and blue bands in a spectrally sensitive band of the imaging-acquisition portion.

16. An endoscope system according to claim 1, wherein the light-source portion is disposed outside the body cavity.

17. An endoscope system, at least a portion of which is inserted inside a body cavity of a living organism for acquiring images of an acquisition object inside the body cavity, comprising:
a light-source portion configured to emit excitation light and illumination light having a different spectral characteristic from the excitation light, which are radiated towards the acquisition object;
an optical system configured to transmit the excitation light or the illumination light toward the acquisition object;
an image-acquisition portion, disposed in the portion inserted inside the body cavity and capable of acquiring fluorescence emitted from the acquisition object, due to the excitation light, and reflected light of the illumination light reflected at the acquisition object;
a variable-spectrum unit, disposed in a light path between the image-acquisition portion and a tip of the portion inserted inside the body cavity and capable of changing the wavelength of light incident on the image-acquisition portion from the acquisition object by varying the spectral characteristics thereof; and
a control portion configured to control the light-source portion, the variable-spectrum unit, and the image-acquisition portion in association with the spectral characteristics of the excitation light and the illumination light that the light-source portion emits, the spectral characteristics of the variable-spectrum unit, and an exposure level of the image-acquisition portion.

18. An endoscope system according to claim 17, wherein the fluorescence is light emitted by exciting, with the excitation light, a fluorescent agent which binds with a specific substance present in the acquisition object or a fluorescent agent which is accumulated in an organ of the living organism, and is light in a band from red to near-infrared.

19. An endoscope system according to claim 17, wherein the fluorescence is light in the visible band emitted by exciting, with the excitation light, a substance originally present in the acquisition object.

20. An endoscope system according to claim 17, wherein the variable-spectrum unit is configured to enable selective switching between a first state in which the fluorescence emitted from the acquisition object is allowed to be incident on the image-acquisition portion and a second state in which the fluorescence emitted from the acquisition object is prevented from being incident on the image-acquisition portion.

21. An endoscope system according to claim 17, wherein, in the first and second states, the variable-spectrum unit has a common transmission band in the spectral characteristics thereof.

22. An endoscope system according to claim 21, wherein the common transmission band includes at least part of a green to blue band in a visible band formed of red, green, and blue.

23. An endoscope system according to claim 17, wherein the control portion time-division switches between the excitation light and the illumination light to be emitted from the light-source portion.

24. An endoscope system according to claim 17, wherein the variable-spectrum unit includes optical members mutually opposing each other with a gap therebetween, and the spectal transmittance is varied by changing the size of the gap between the optical members.

25. An endoscope system according to claim 17, wherein the control of the exposure level of the image-acquisition portion by the control portion is performed by light-level control of the light-source portion or exposure adjustment of the image-acquisition portion in response to the switching of the emitted irradiation light from the light-source portion.

26. An endoscope system, at least a portion of which is inserted inside a body cavity of a living organism for acquiring images of an acquisition object inside the body cavity, comprising:
a light-source portion configured to emit a plurality of types of irradiation light having different spectral characteristics, which are radiated towards the acquisition object;
an optical system configured to emit the irradiation light from the light-source portion towards the acquisition object;
image-acquisition means, disposed in the portion that is inserted inside the body cavity and capable of acquiring fluorescence emitted from the acquisition object due to irradiation with the plurality of types of irradiation light, and light with a different wavelength band from the fluorescence;
variable-spectrum means, disposed in a light path between the image-acquisition means and a tip of the portion inserted inside the body cavity and capable of changing the wavelength band of light incident on the image-acquisition means from the acquisition object by varying the optical characteristics thereof; and
control means for controlling the light-source portion, the variable-spectrum means, and the image-acquisition means in association with the spectral characteristics of the irradiation light which the light-source portion emits, the spectral characteristics of the variable-spectrum means, and an exposure level of the image acquisition means.

27. An endoscope system, at least a portion of which is inserted inside a body cavity of a living organism for acquiring images of an acquisition object inside the body cavity, comprising:
a light source portion configured to emit excitation light and illumination light having different spectral characteristics from the excitation light, which are radiated towards the acquisition object;
an optical system configured to transmit the excitation light or the illumination light towards the acquisition object;
image-acquisition means, disposed in the portion inserted inside the body cavity and capable of acquiring fluorescence emitted from the acquisition object, due to the excitation light, and reflected light of the illumination light which at the acquisition object;
variable-spectrum means, disposed in a light path between the image-acquisition means and a tip of the portion inserted inside the body cavity and capable of changing the wavelength band of light incident on the image-acquisition means from the acquisition object by varying the spectral characteristics thereof; and
control means for controlling the light-source portion, the variable-spectrum means, and the image-acquisition means in association with the spectral characteristics of the excitation light and the illumination light that the light-source portion emits, the spectral characteristics of the variable-spectrum means, and an exposure level of the image-acquisition means.
